(19) United States
(12) Patent Application Publication
(10) Pub. No.: US 2009/0105665 A1
(43) Pub. Date: Apr. 23, 2009

(54) Drip infusion IV catheter check cover set

(76) Inventors: Quincy Wang, Taichung City (TW); Yu-Shu Liao, Taichung City (TW)

Correspondence Address: TROXELL LAW OFFICE PLLC, SUITE 1404, 5205 LEESBURG PIKE, FALLS CHURCH, VA, 22041, US

(21) Appl. No.: 11/976,305

(22) Filed: Oct. 23, 2007

(51) Int. Cl. A61M 5/168

(52) U.S. Cl. 604/247

(57) ABSTRACT

A check sealing set of intravenous injection catheter for drip infusion to seal up a needle retained in human body includes a check body, a locking seat, and a mobile cover; the check body related to an arc body provided with a retaining edge and a check seam disposed below the retaining edge; the locking seat related to a tube seat having on one end disposed with a locking slot and a groove on its outer circumference; the mobile cover has its neck ring to engage the groove of the locking seat so to merely cover up an exposed end of the locking seat; the check set is connected to an IV set when assembled with a terminal of the IV catheter for preventing blood from flowing back to a tube (it is impossible for the prior art of drip infusion to provide the check purpose); the tube is plugged off when the drip infusion is not required for the check cover to serve a protection cover to allow direct insertion through a rubber pad if subsequent drip infusion or adding medicine into the fluid, thus to avoid repeated penetration of the catheter due to peripheral IV site failure and solve the problem of storage of the catheter cover when removed.

### BACKGROUND OF THE INVENTION

(a) Field of the Invention

The present invention is related to a check cover set, and more particularly, to a check set to control injection fluid and transfusion blood (including pulsation blood) to advance only in one direction (i.e., preventing backflow) by means of a check body and to seal up a tube connection end and needle seat end when not used by means of a mobile cover located to a locking seat; accordingly, it is not necessary to remove the cover in drip infusion or adding medicine into a drip chamber for preventing backflow of the fluid and blood and allowing safer and more convenient drip infusion.

(b) Description of the Prior Art

An IV catheter is usually used in drip infusion to be retained on human body with its rear end connected to a tube for guiding a fluid to drop into human body. Upon completing or suspending the drip infusion, an IV lock or IV cap is applied to cover up a needle of the catheter and the injection needle is removed so to prevent risk of blood backflow. However, in subsequent drip infusion or adding medications, the IV lock or IV cap must be removed before injection with the tube resulting in waste of the IV lock or IV cap, and the method of adding medication by direct penetration with the catheter easily result in peripheral IV site failure, increasing opportunity of infection by the patient, while exposing nursing staff to be infected due to contact blood of the patient. The IV catheter of the prior art is not convenient to use and also threatens the health of the nursing staff.

Most of patients (disregarding how severe the condition is) entering the hospital for care have some form of intravenous therapy during their stay in hospital to receive intensive drip infusion of nutritional fluids, blood products or medications. However, the catheter cover has to be frequently opened or closed resulting in failure of insertion site to warrant repeated insertions. In serious case, the patient may be infected with septicemia while presenting many problems to the nursing staff in providing drip infusion to the patient.

It is not uncommon that medications will be added in the course of drip infusion and it is usually done by injection of the medications into the tube with a syringe. For each addition, one syringe must be consumed and disposed of so to avoid infection by bacteria. This is not only a waste of resources but also presents the problem of disposal of the needle.

### SUMMARY OF THE INVENTION

The primary purpose of the present invention is to provide a check cover set for catheters applied in drip infusion that prevents backflow of medication fluid and minimizing consumption of syringe. The present invention is simple in construction, saving the use of a syringe when addition of medications warrants, minimizing inconvenience and disturbance in the disposal of the needle while providing readily and economic use benefits.

To achieve the purpose, the present invention provides a needle seat to prevent blood backflow for safety use and a cover to cover up an end of injection fluid to prevent infection by bacteria so to directly retain the catheter inserted into the vein waiting for next round of drip infusion without repeated insertion and removing the catheter for minimizing damage to the peripheral site of the patient.

When assembled to a Y shaped tube of a drip infusion device, the tube is permitted to options of having single or double medication fluid so to skip the use of a syringe for adding medications and to immediately apply the cover in case of single medication injection to prevent backflow of the medication fluid and minimize consumption of syringe.

### BRIEF DESCRIPTION OF THE DRAWINGS

FIG. 1 is an exploded view of the present invention.

FIG. 1A is a perspective view showing a check body in the present invention.

FIG. 2 is a perspective view of the present invention.

FIG. 3 is a schematic view showing an operating status of the present invention when applied in drip infusion.

FIG. 4 is a schematic view showing another operating status of the present invention when applied in addition of medication during intravenous injection.

FIG. 5 is a schematic view showing another operating status yet of the present invention when a cover is closed after completion of the intravenous injection.

FIG. 6 is a schematic view showing another construction of a mobile cover.

FIG. 6A is a schematic view showing an operating status of the mobile cover taken from FIG. 6.

### DETAILED DESCRIPTION OF THE PREFERRED EMBODIMENTS

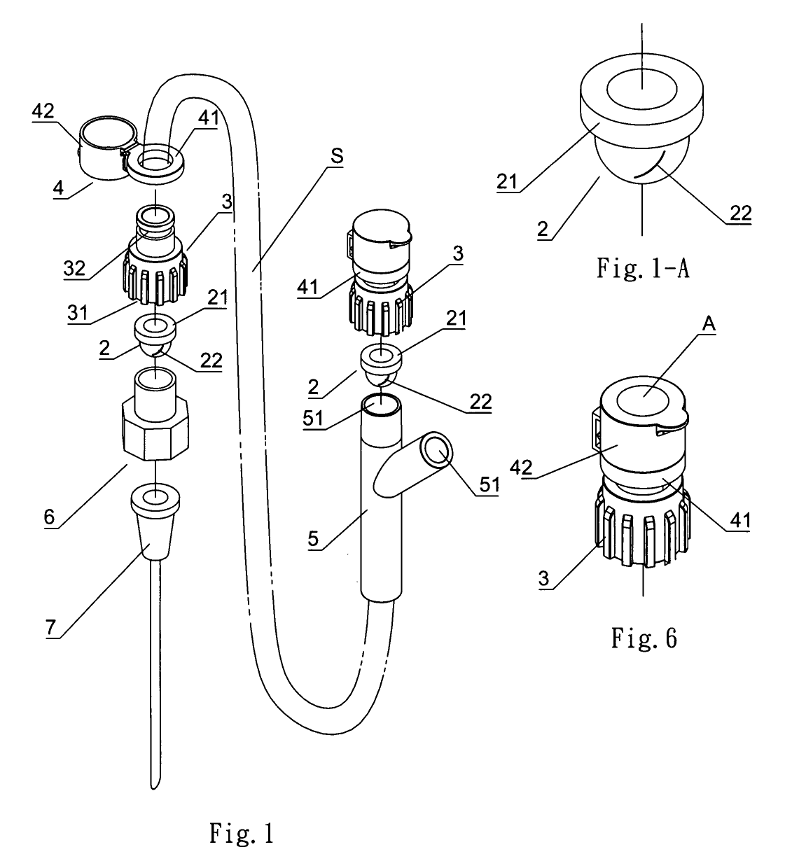

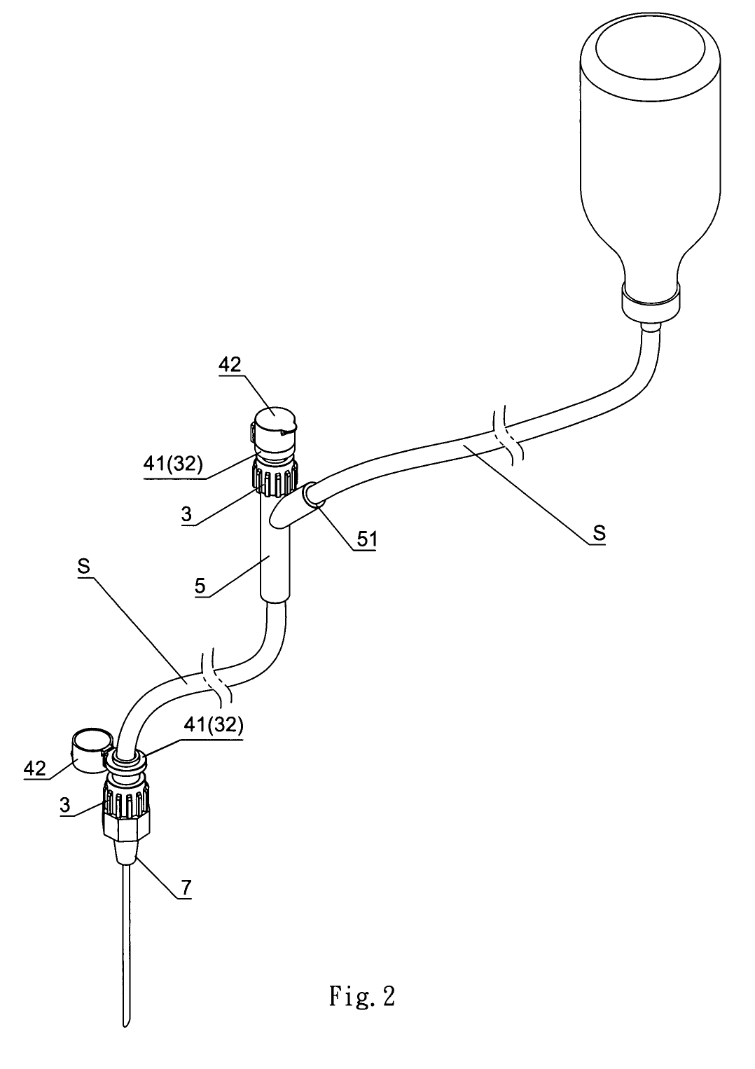

Referring to FIGS. 1, 1A, and 2, a preferred embodiment of the present invention is essentially comprised of a check body 2, a locking seat 3, and a mobile cover 4.

The check body 2 is related to an arc shaped body provided with a retaining ring 21, and a check slot 22 is disposed on an end of the check body 2 at where below the retaining ring 21. The locking seat 3 is related to a tube seat disposed at one end a locking slot 31 and a groove 32 in proper depth is disposed on an outer circumference of the locking seat 3. The mobile cover 4 is provided with a neck ring 41 to engage the groove 32 of the locking seat 3 for a sealing body 42 to cover up an exposed end of the locking seat.

Accordingly, a multi-purpose check cover set of catheter for drip infusion of the present invention provides control function of injection of fluid to advance in one direction and prevents blood or medication fluid from backflow through the check slot 22 and protection function by covering up the catheter when not used.

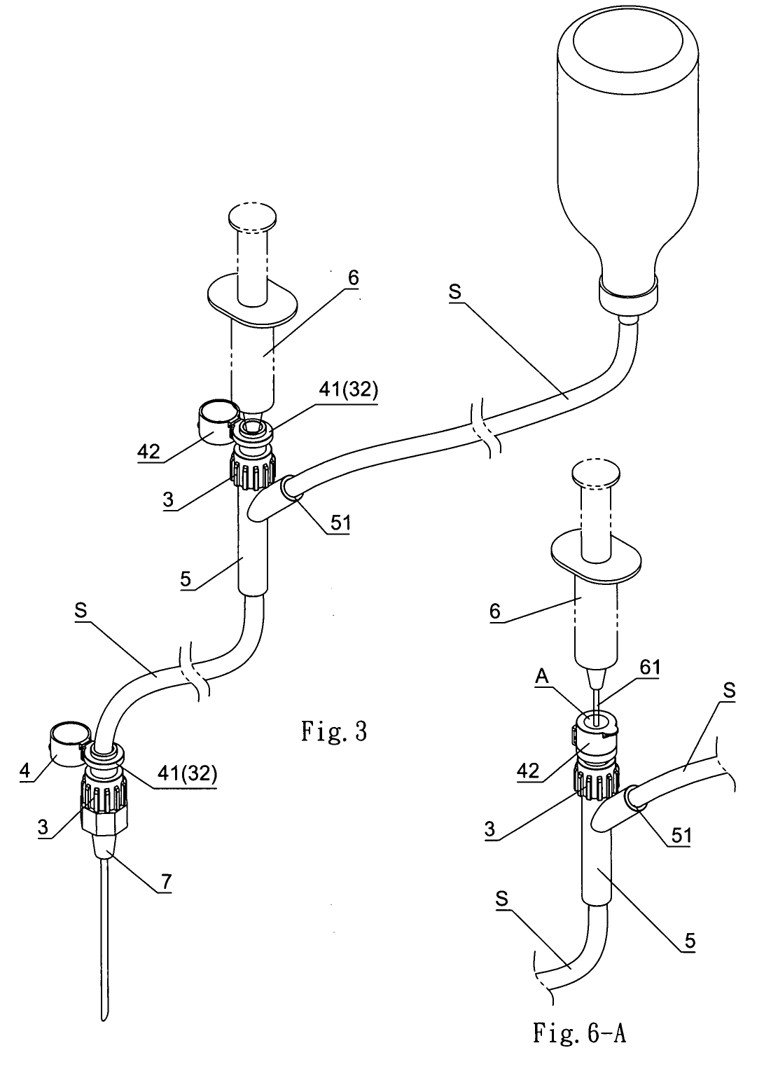

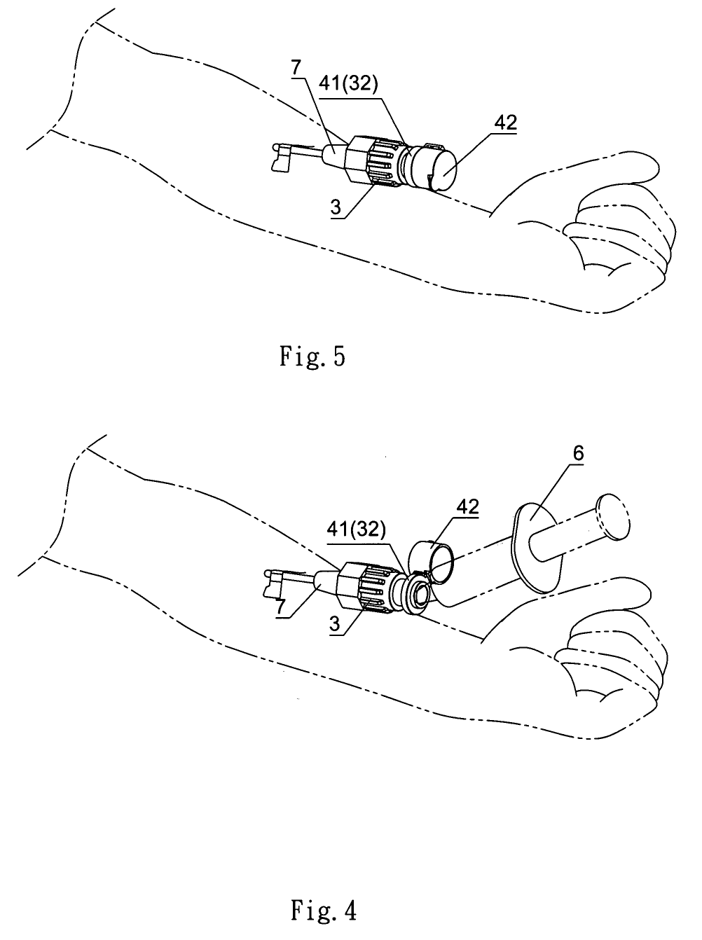

Now referring to FIGS. 3, 4, and 5 when the present invention is applied in drip infusion and intravenous injection, the present invention is assembled to a Y-shaped tube 5 extended from a plastic tube S for the Y-shaped tube 5 to select one-way or two-way (i.e., injection of two different types of solutions at the same time); meanwhile a syringe 6 without needle is inserted with its tip into an inner tube of the locking seat 3 before inject a medication fluid as prescribed to complete the addition of medication fluid without needle as illustrated in FIG. 3. In case of one-way injection, an opening 51 of a branch of the Y-shaped tube when not used is sealed by the sealing body 42. When the present invention is applied in conjunction with a needle seat 7 in intravenous injection, the present invention provides a secured site on a forearm of the patient to facilitate connection with the plastic tube S at any time as desired to achieve the same purpose of addition of medication fluid without needle as illustrated in FIG. 4. When the needle seat 7 is not used, it is immediately covered up by the sealing body 42 for zero contamination and safe use as illustrated in FIG. 5.

As illustrated in FIGS. 6 and 6A, an additional rubber pad A in proper size is disposed at a center of the sealing body 42 of the mobile cover 4 so that when the mobile cover 4 is not used, the sealing body 42 is opened up to receive direct insertion of a tip 61 of the syringe 6 through the rubber pad A for adding medication fluid in drip infusion of intravenous injection as illustrated in FIG. 6-A.

I claim:

1. A check cover set of catheter for drip infusion comprising a check body, a locking seat, and a mobile cover; the check body being related to an arc shaped body provided with a retaining ring and a check slot being disposed on an end of the check body at where below the retaining ring; the locking seat related to a tube seat being disposed at one end a locking slot and a groove in proper depth being disposed on an outer circumference of the locking seat; the mobile cover being provided with a neck ring to engage the groove of the locking seat for a sealing body to cover up an exposed end of the locking seat; the check set being mounted to a needle seat and a Y-shaped drip tube to facilitate injection of two types of medication fluids and to prevent backflow; the Y-shaped tube being located by the cover an opening of a branch of the Y-shaped tube not used and retaining the catheter in position to avoid repeated insertion at injection site, prevent blood backflow, and allow synchronous application of drip infusion solution and medication fluid for achieving the purpose of zero contamination.

2. The check cover set of catheter for drip infusion as claimed in claim 1, wherein a rubber pad to facilitate insertion of a needle is disposed at a center of the sealing body of the mobile cover.

3. The check cover set of catheter for drip infusion as claimed in claim 1, wherein the mobile cover is made an integrated part extending from the locking seat.
